(19) United States
(12) Patent Application Publication
(10) Pub. No.: US 2005/0019880 A1
(43) Pub. Date: Jan. 27, 2005

(54) Method of enhancing levels of polyunsaturated fatty acids in thraustochytrid protists

(75) Inventors: S. Raghukumar, Goa (IN); Ruchi Jain, Goa (IN)

Correspondence Address: FOLEY AND LARDNER;SUITE 500, 3000 K STREET NW, WASHINGTON, DC, 20007, US

(73) Assignee: COUNCIL OF SCIENTIFIC AND INDUSTRIAL RESEARCH

(21) Appl. No.: 10/812,389

(22) Filed: Mar. 30, 2004

Related U.S. Application Data
(60) Provisional application No. 60/458,443, filed on Mar. 31, 2003.

(51) Int. Cl. C12P 7/64; C12N 1/10

(52) U.S. Cl. 435/134; 435/258.1

(57) ABSTRACT

The present invention relates to a method for enhancing levels of polyunsaturated fatty acids in thraustochytrid protists and more particularly to a method of enhancing levels of docosahexaenoic acid and eicosapentaenoic acid in cells of thraustochytrid protist belonging to the genera Schizochytrium, Thraustochytrium and Aplanochytrium deposited at The Microbial Type Culture Collection (MTCC), Institute of Microbial Technology, Chandigarh, India under the accession numbers MTCC 5121, MTCC 5122 and MTCC 5123 respectively by growing the same in a medium with increased viscosity, whereby the cells thus enriched in the said polyunsaturated fatty acids (PUFAs) can then be utilized successfully in various beneficial applications that require polyunsaturated fatty acids, such as in animal feeds, human nutrition and extraction of the PUFAs for nutritional supplementation.

### FIELD OF THE PRESENT INVENTION

The present invention relates to a method for enhancing levels of polyunsaturated fatty acids in thraustochytrid protists. More particularly, the present invention relates to a process for enhancement of the polyunsaturated fatty acids, docosahexaenoic acid and eicosapentaenoic acid in cells of thraustochytrid protist belonging to the genera Thraustochytrium, Schizochytrium and Aplanochytrium, by growing the same in a medium with increased viscosity. The cells thus enriched in the said polyunsaturated fatty acids (PUFAs) can then be utilized more successfully than cells that are not enriched in the PUFAs, in various beneficial applications that require polyunsaturated fatty acids, such as in animal feeds, human nutrition and extraction of the PUFAs for nutritional supplementation.

### BACKGROUND AND PRIOR ART REFERENCES

Fatty acids are constituents of lipids, which are required by all living organisms for growth, survival and reproduction. Among the fatty acids, saturated fatty acids are those with a chemical structure in which the carbon atoms are connected to each other only by single bonds and contain no double bonds. Unsaturated fatty acids are those in which one or more of the carbon atoms are connected to each other by double bonds. Polyunsaturated fatty acids, termed as PUFAs hereafter, are those in which more than one such double bonds are found. Among the PUFAs, two are considered extremely essential in the health of animals and human beings. These are the docosahexaenoic acid and eicosapentaenoic acid, termed DHA and EPA hereafter. The molecular structure of both DHA and EPA is such that the first double bond follows the third carbon atom from the methyl end of the fatty acid structure. Therefore, these are also called omega-3 PUFAs. DHA contains 22 carbon atoms, between which six double bonds are found. EPA contains 20 carbon atoms, between which five double bonds occur. Both DHA and EPA have been shown to be important for human health and in animal nutrition. In human health, DHA and EPA have been shown to be important in brain development in children, prevention of atherosclerosis, prevention of night blindness, neurological disorders and even for possible prevention of cancer (Bajpai, P. and P. K. Bajpai. 1993. Journal of Biotechnology 30: 161-183; Barclay, W. R. et al. 1994. Journal of Applied Phycology 6: 123-129; Singh A. and O. P. Ward. 1997.

Advances in applied microbiology, 45: 271-312). These two omega-3 PUFAs have been shown to enhance growth and reproduction in crustacean animals, such as prawns, which are very important as aquaculture animals for human consumption (Harrison, K. E. 1990. Journal of Shellfish Research 9: 1-28). Incorporation of DHA and EPA in human and animal feeds is therefore considered important. DHA and EPA levels of thraustochytrid protists can be enhanced beyond their natural levels by growing the cells in a medium with increased viscosity, as detailed in the present invention, and their cells can be of still better use as supplement to human nutrition and as feed for animals compared to presently known processes. Thraustochytrids can be cultivated on a large scale, using well-established fermentation techniques. Cells thus obtained can be used as animal feeds, by suitably processing and preserving their cells, such as by spray-drying and freezing. The cell biomass, enhanced in the omega-3 fatty acids can also be harvested and DHA and EPA extracted in a pure form. These may be used to supplement human food that is poor in these essential omega-3 PUFAs.

One major source of EPA and DHA for human consumption is in the form of fish oil. However, fish oil has the disadvantage of an odour, which is disagreeable to many human consumers. Fish containing DHA and EPA are also highly seasonal and variable in their omega-3 PUFA contents. Besides, most of the fish oil is hydrogenated and the omega-3 PUFAs are destroyed. For these reasons, micro-organisms containing EPA and DHA, which can be cultivated on a large scale are considered suitable for use in human nutrition and animal feeds (Bajpai, P. and P. K. Bajpai. 1993. Journal of Biotechnology 30: 161-183). Several single-celled plants, the algae, contain high levels of EPA and DHA and have been considered for the said purposes. Refer to A. Singh (Singh, A. and O. P. Ward, 1997. Advances in Applied Microbiology 45: 272-312).

Microorganisms can be easily cultivated on a large scale using cheap nutrients. Several groups of microorganisms contain high amounts of EPA and DHA. Such organisms can be used directly as feed, or the said PUFAs can be extracted from them for further use. Search for microorganisms containing high amounts of DHA and EPA has shown that thraustochytrid protists contain some of the highest amounts of DHA and EPA. Thraustochytrids are already considered of commercial importance. Their cells are used in animal feeds or for extraction of PUFAs for commercial use (Lewis, T. E. et al., 1999, The Biotechnological potential of thraustochytrids. Marine Biotechnology 1: 580-587; U.S. Pat. No. 6,451,567 of 17 Sep. 2002).

The Japanese Patent No. 9633263 (1996) describes a strain of a thraustochytrid for application in the food industry such as food-additives, nutritional supplements, as additives for infant milk formula, feedstuffs and drug additives. The strain contains at least 2% of dry wt as DHA. Japanese patent No. 980 3671 (1998) describes the production by fermentation of DHA and PUFA, docosapentaenoic acid (DPA) from lipids of thraustochytrid protists. Cells of thraustochytrid protists may be directly used as feed in aquaculture (U.S. Pat. No. 5,908,622 of 1 Jun. 1999). Alternatively, DHA and EPA may be extracted from thraustochytrid cells (Japanese Patents JP 103105555 of 24 Nov. 1998 and JP 10310556 of 12 May 1997). U.S. Pat. No. 5,340,594 describes a process for production of whole-celled or extracted microbial products using thraustochytrid protists with a high concentration of the omega-3 PUFAs. There is a potential for the use of thraustochytrid protists as human nutraceuticals (Application A428 of Australia New Zealand Food Authority (ANZFA), 12 Dec. 2001).

The production of DHA and EPA from thraustochytrid protists requires that they be grown in suitable conditions in fermentors, to yield commercially useful amounts of the PUFAs. Several research papers and patents address the issue of providing suitable conditions for growth and production of DHA and EPA in thraustochytrids. U.S. Pat. No. 5,340,742 discloses a process for growing the thraustochytrid protists in defined media suitable for their growth. U.S. Pat. No. 6,461,839 of 8 Oct. 2002 provides a method of producing PUFAs in Labyrinthula sp., using a culture medium containing oil or fatty acid as a carbon source. Yokochi et al., (1999; Applied Microbiology and Biotechnology, 49, 72-76) describe salinity, temperature, carbon source, oil and nitrogen sources for production of high amounts of DGA in the thraustochytrid Schizochytrium limacinum. Optimal pH and medium ingredients have also been described for Thraustochytrium aureum (Iida T., Journal of Fermentation and Biogengineering, 81: 76-78).

All the above patents relate to screening numerous thraustochytrid cultures, selecting the strain with the highest DHA and EPA content, prepare mutant strains of these and cultivate such strains under optimal culture conditions for commercial production. Besides, the above mentioned prior art patents reject a large number of strains, which might have only moderate DHA and EPA concentrations.

U.S. Pat. No. 6,410,282 which was earlier filed by the Inventors and which has been assigned to Council of Scientific and Industrial Research provides a method for enhancing polyunsaturated fatty acids in the thraustochytrid Ulkenia radiata Gaertner, by which the ingredients of the culture medium need not be altered, but by which only the viscosity of the medium is increased by addition of polyvinyl pyrrolidone (PVP). This patent is restricted to only one genus and species of thraustochytrids. As it is commonly known to a person of ordinary skill in the art, it cannot be assumed that a process which is applicable to a particular one genus and species of a microorganism can be applied to another genus and species of the microorganism. Further, the Inventors would like to highlight here that the process described in U.S. Pat. No. 6,410,282 was specifically designed keeping in view only one genus and species i.e. Ulkenia radiata and does not include other species. He aforesaid U.S. Patent also fails to provide any suggestion that the process described could be adopted for other species of thraustochytrid. In view of the above, it should not be assumed that the process of the present invention is obvious or lacking novelty over the process described in the aforesaid U.S. Patent. The present invention aims to extend the above U.S. Pat. No. 6,410,282 on Ulkenia radiata to include other genera of thraustochytrids, and to increase the DHA and EPA levels in these species independently of nutrients and culture conditions, so that they will provide still higher commercial yields of the said PUFAs. In the present invention, even strains with moderate amounts of DHA and EPA can be made to produce higher amounts of these PUFAs by growing them in media with increased viscosity.

### OBJECTS OF THE PRESENT INVENTION

The main object of the present invention is to enhance the amounts of PUFAs in thraustochytrid protists, which obviates the drawbacks as detailed above.

Another object of the invention is to make strains of thraustochytrids to produce higher amounts of DHA and EPA than they normally produce using optimal nutrient conditions. Yet another object of the present invention is to enhance the levels of these fatty acids by growing the cultures of thraustochytrid protists in a medium with increased viscosity.

### SUMMARY OF THE PRESENT INVENTION

The present invention relates to a method for enhancing levels of polyunsaturated fatty acids in thraustochytrid protists and more particularly to a method of enhancing levels of docosahexaenoic acid and eicosapentaenoic acid in cells of thraustochytrid protist belonging to the genera Schizochytrium, Thraustochytrium and Aplanochytrium deposited at The Microbial Type Culture Collection (MTCC), Institute of Microbial Technology, Chandigarh, India under the accession numbers MTCC 5121, MTCC 5122 and MTCC 5123 respectively by growing the same in a medium with increased viscosity, whereby the cells thus enriched in the said polyunsaturated fatty acids (PUFAs) can then be utilized successfully in various beneficial applications that require polyunsaturated fatty acids, such as in animal feeds, human nutrition and extraction of the PUFAs for nutritional supplementation.

### DETAILED DESCRIPTION OF THE PRESENT INVENTION

Accordingly, the present invention provides a method for enhancing levels of docosahexaenoic acid and eicosapentaenoic acid in thraustochytrid protist, comprising the steps of:
(a) inoculating the thraustochytrid protist belonging to the genera Schizochytrium deposited at The Microbial Type Culture Collection (MTCC), Institute of Microbial Technology, Chandigarh, India under the accession number MTCC 5121 or Thraustochytrium deposited at The Microbial Type Culture Collection (MTCC), Institute of Microbial Technology, Chandigarh, India under the accession number MTCC 5122 or Aplanochytrium deposited at The Microbial Type Culture Collection (MTCC), Institute of Microbial Technology, Chandigarh, India under the accession number MTCC 5123 in a culture medium and growing the same for about 2 days at 25° C. to 30° C.;
(b) obtaining the cultures thus grown for use as inoculum and inoculating a medium having increased viscosity using the same;
(c) growing the thraustochytrid protist culture in the culture medium having increased viscosity of step (b) for 2 to 5 days at 25° to 30° C., and
(d) harvesting the cells by centrifugation and extracting the enhanced amounts of docosahexaenoic acid and eicosapentaenoic acids from the cells.

In an embodiment of the present invention wherein in step (a), the culture medium used comprises peptone in the range of 0.5% Wt. to 1.5% Wt.; yeast extract in the range of 0.01% Wt. to 0.1% Wt.; glucose in the range of 0.01% to 1.0% Wt.; and sea water of about 100 ml. In another embodiment of the present invention wherein in step (b), the culture medium used comprises peptone in the range of 0.5% Wt. to 1.5% Wt.; yeast extract in the range of 0.01% Wt. to 0.1% Wt.; glucose in the range of 0.01% to 1.0% Wt.; polyvinyl pyrrolidone in the range of 0.5% Wt. to 1.5% Wt.; and sea water of about 100 ml.

In yet another embodiment of the present invention, wherein the culture comprises 1.5% peptone; 0.1% yeast extract; 1.0% glucose; 1.0% polyvinyl pyrrolidone and 100 ml sea water.

In still another embodiment of the present invention, wherein said culture medium comprises 1.5% peptone.

In one more embodiment of the present invention, wherein said culture medium comprises 0.1% yeast extract.

In one another embodiment of the present invention, wherein said culture medium comprises 1.0% glucose.

According to the present invention, culture of a candidate species of the thraustochytrid fungus, which contains the omega-3 PUFAs DHA and EPA is first inoculated into a liquid nutrient medium. Strains of fungi belonging to Thraustochytrium sp. of thraustochytrid fungi, such as those with the American Type Culture Collection having ATCC Numbers 18906, 18907, 20890, 20891, 20892, 26185, or belonging to Thraustochytrium roseum Gaertner having accession number No. 28210 or belonging to Thraustochytrium aureum Goldstein having accession number No. 34304 may be used.

A suitable medium for example, is one containing peptone, yeast extract, glucose and sea water. Any other medium that supports good growth of the fungus also may be used. The culture is grown for 2 days at a room temperature ranging from 25 to 30 degree C. This culture is used as the inoculum and used to inoculate a medium with enhanced viscosity. The compound that is added to increase viscosity may be one of the common polymers, such as dextran or polyvinyl pyrrolidone (PVP) that are not utilised as nutrients by the organisms, but only contribute to increasing the medium viscosity. For example, polyvinyl pyrrolidone (PVP) is a water-soluble polymer of basic nature (McGraw-Hill Encyclopaedia of Science and Technology, Vol. 10, 1982). PVP is commonly used to increase fluid viscosity and is a suitable agent for this purpose (Podolsky, R. D. and R. B. Emlet, 1993. Journal of experimental biology 176:207-221).

In the present example, PVP at concentrations of 0.1 to 1.0% are added to the medium. Cultures may be grown in flasks on a rotary shaker in the laboratory or in a fermentor when large-scale cultivation is required. The culture is allowed to grow at room temperature of 25 to 30 degree C. or any temperature at which the particular strain grows best. After a suitable period, for example 2 to 7 days growth, cells from the culture are harvested. This may be done by any appropriate method, such as centrifugation, continuous flow centrifugation, filtration etc. Cells thus obtained may be used for all applications that require thraustochytrid cells. Such use may include cell biomass for animal feed, human food supplement or extraction of pure DHA and EPA.

Accordingly, the present invention provides a method for enhancing levels of polyunsaturated fatty acid levels in thraustochytrid fungi, using culture media supplemented with polyvinyl pyrrolidone (PVP) to increase viscosity and which comprises: Step a: Providing a thraustochytrid protist belonging to the genera Thraustochytrium, Schizochytrium or Aplanochytrium (formerly called Labyrinthuloides); Step b: Inoculating the above said strain in a culture medium; Step c: Growing the culture for 2 days at a temperature ranging from 25 to 30 C; Step d: Obtaining the cultures for use as inoculum using the above said medium to inoculate a medium with different concentrations of polyvinyl pyrrolidne (PVP); Step e: Growing the culture separately for 2 to 5 days at a temperature ranging from 25 to 30 C; Step f: Harvesting the cells from the above culture by centrigugation and extracting the enhanced amounts of docosahexaenoic acid (DHA) and eicosapentaenoic acids (EPA).

In an embodiment of the present invention, the viscosity of the medium is increased by incorporating polyvinyl pyrrolidone (PVP) at a concentration of 0.5 to 1.0%.

In yet another embodiment of the present invention, a process is provided to enhance the levels of the PUFAs in cells of thraustochytrid protists.

In yet another embodiment of the present invention, the PUFAs that are enhanced are DHA and EPA.

In yet another embodiment of the present invention, DHA and EPA are enhanced in cells of thraustochytrid fungi by growing the cultures in a medium with increased viscosity.

In yet another embodiment of the present invention, the increase in viscosity is provided by incorporating a substance that is not utilised as nutrients such as polyvinyl pyrrolidone (PVP) at a concentration of 1.0%.

The present invention thus relates to a process to enhance the levels of the omega-3 PUFAs, DHA and EPA. By this process, strains of cultures of thraustochytrids can be made to produce higher levels of these PUFAs than they do under other conditions. Besides, even strains that contain only moderate quantities of these PUFAs under normal conditions can be made to produce greater amounts within their cells.

The invention is described in detail hereafter with reference to the accompanying drawings which are provided merely to illustrate this invention.

### BRIEF DESCRIPTION OF THE ACCOMPANYING DRAWINGS

In the drawings accompanying the specification,

FIG. 1 represents the DHA contents of a thraustochytrid strain corresponding in its morphology and life cycle to the genus Schizochytrium Goldstein and Belsky, deposited at The Microbial Type Culture Collection (MTCC), Institute of Microbial Technology, Chandigarh, India under the accession number MTCC 5121, when grown in a liquid nutrient culture medium.

FIG. 2 represents the EPA contents of a thraustochytrid strain corresponding in its morphology and life cycle to the genus Schizochytrium Goldstein and Belsky, deposited at The Microbial Type Culture Collection (MTCC), Institute of Microbial Technology, Chandigarh, India under the accession number MTCC 5121, when grown in a liquid nutrient culture medium.

FIG. 3 represents the DHA contents of a thraustochytrid strain corresponding in its morphology and life cycle to the species Thraustochytrium Sparrow, deposited at The Microbial Type Culture Collection (MTCC), Institute of Microbial Technology, Chandigarh, India under the accession number MTCC 5122, when grown in a liquid nutrient culture medium.

FIG. 4 represents the EPA contents of a thraustochytrid strain corresponding in its morphology and life cycle to the species Thraustochytrium Sparrow, deposited at The Microbial Type Culture Collection (MTCC), Institute of Microbial Technology, Chandigarh, India under the accession number MTCC 5122, when grown in a liquid nutrient culture medium.

FIG. 5 represents the DHA contents of a thraustochytrid strain corresponding in its morphology and life cycle to the species Ulkenia, deposited at National Institute of Biosciences and Human Technology, Japan under the accession number AB22115, when grown in a liquid nutrient culture medium.

FIG. 6 represents the EPA contents of a thraustochytrid strain corresponding in its morphology and life cycle to the species Ulkenia, deposited at National Institute of Biosciences and Human Technology, Japan under the accession number AB22115, when grown in a liquid nutrient culture medium.

FIG. 7. represents the DHA contents of a thraustochytrid corresponding in its morphology and life cycle to the species Aplanochytrium, deposited at The Microbial Type Culture Collection (MTCC), Institute of Microbial Technology, Chandigarh, India under the accession number MTCC 5123, when grown in a liquid nutrient culture medium.

The following examples are given by way of illustrations of the present invention and therefore, should not be construed to limit the scope of the present invention.

### EXAMPLES

### Example 1

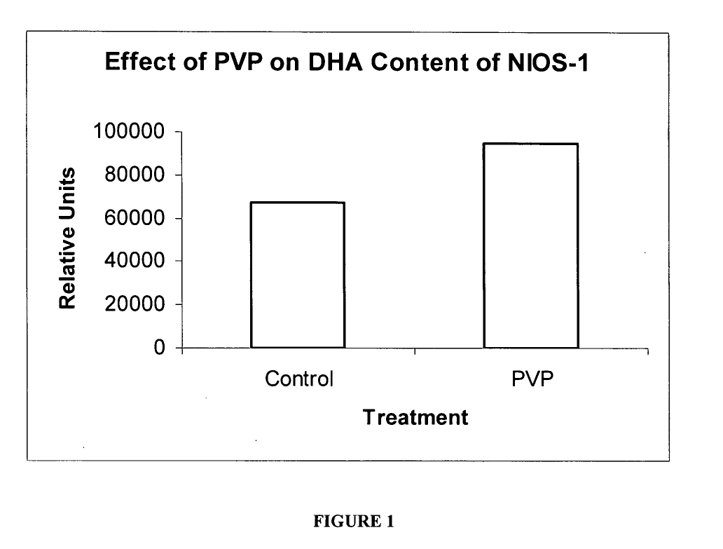

A culture of a thraustochytrid, belonging to strain #NIOS-1 was inoculated into 100 ml of a culture medium containing: gelatin peptone—1.5% Wt.; Yeast extract—0.1% Wt.; Glucose—1.0% Wt. and sea water—100 ml. The cultures were grown for 2 days on a shaker at room temperature of 25-30° C. These cultures were used as innoculum for the experiment. A set of cultures was set up using a medium with the same composition as above, containing an addition of 1.0% polyvinyl pyrrolidone. The experiment was carried out by adding 10 ml of the inoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media with increased viscosity by adding PVP contained nearly 0.5 times more DHA than those grown in a medium without increased PVP (FIG. 1).

### Example 2

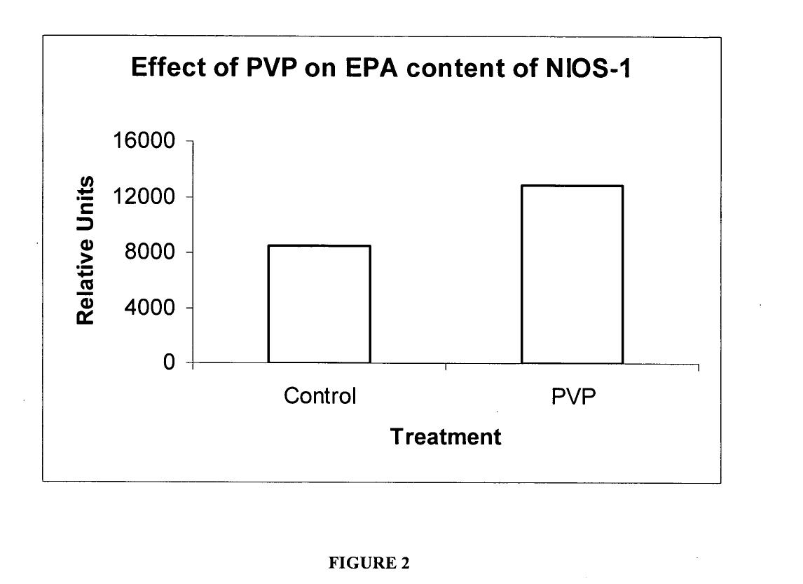

A culture of a thraustochytrid, belonging to strain #NIOS-1 was inoculated into 100 ml of a culture medium containing: gelatin peptone—1.5% Wt.; Yeast extract—0.1% Wt.; Glucose—1.0% Wt. and sea water—100 ml. The cultures were grown for 2 days on a shaker at room temperature of 25-30 degree C. These cultures were used as inoculum for the experiment. A set of cultures was set up using a medium with the same composition as above, containing an addition of 1.0% polyvinyl pyrrolidone. The experiment was carried out by adding 10 ml of the inoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media with increased viscosity by adding PVP contained nearly 0.5 times more EPA than those grown in a medium without increased PVP (FIG. 2).

### Example 3

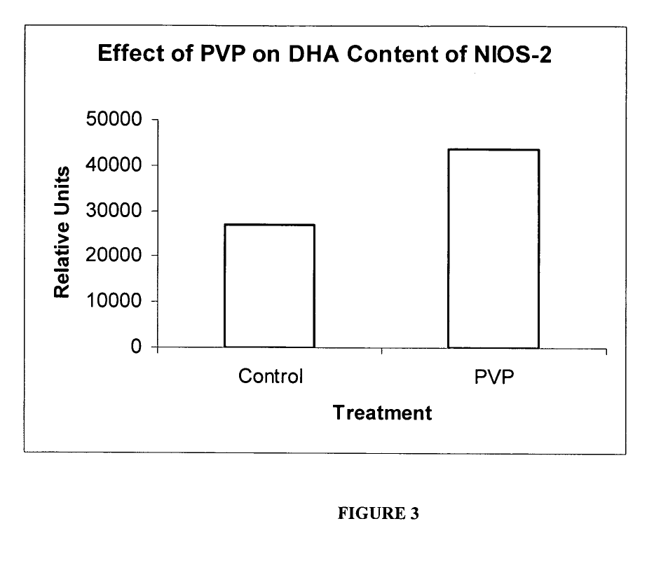

A culture of a thraustochytrid, belonging to strain #NIOS-2 was inoculated into 100 ml of a culture medium containing: gelatin peptone—1.5% Wt.; Yeast extract—0.1% Wt.; Glucose—1.0% Wt. and sea water—100 ml. The cultures were grown for 2 days on a shaker at room temperature of 25-30 degree C. These cultures were used as innoculum for the experiment. A set of cultures was set up using a medium with the same composition as above, containing an addition of 1.0% polyvinyl pyrrolidone. The experiment was carried out by adding 10 ml of the innoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media with increased viscosity by adding PVP contained nearly 0.5 times more DHA than those grown in a medium without increased PVP (FIG. 3).

### Example 4

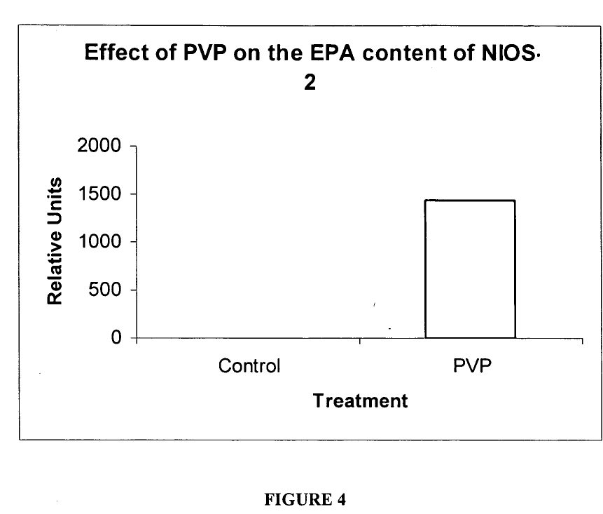

A culture of a thraustochytrid, belonging to strain #NIOS-2 was inoculated into 100 ml of a culture medium containing: gelatin peptone—1.5% Wt.; Yeast extract—0.1% Wt.; Glucose—1.0% Wt. and sea water—100 ml. The cultures were grown for 2 days on a shaker at room temperature of 25-30 degree C. These cultures were used as innoculum for the experiment. A set of cultures was set up using a medium with the same composition as above, containing an addition of 1.0% polyvinyl pyrrolidone. The experiment was carried out by adding 10 ml of the innoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media without increasing viscosity revealed no EPA, while those with increased viscosity by adding PVP contained EPA (FIG. 4).

### Example 5

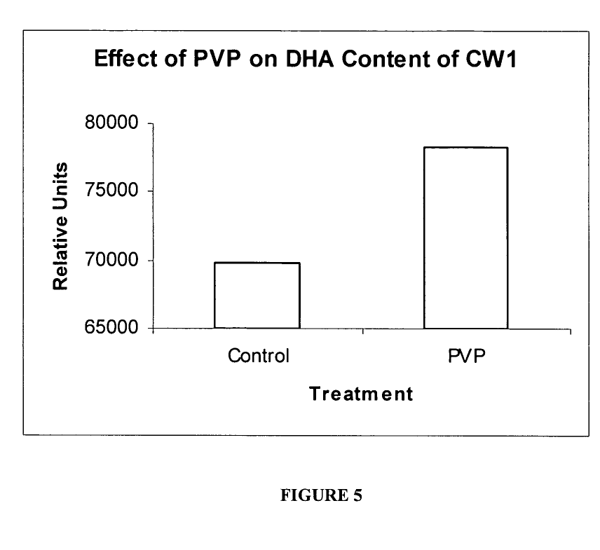

A culture of a thraustochytrid, belonging to strain #NIOS-3 was inoculated into 100 ml of a culture medium containing: gelatin peptone—1.5% Wt.; Yeast extract—0.1% Wt.; Glucose—1.0% Wt. and sea water—100 ml. The cultures were grown for 2 days on a shaker at room temperature of 25-30 degree C. These cultures were used as innoculum for the experiment. A set of cultures was set up using a medium with the same composition as above, containing an addition of 1.0% polyvinyl pyrrolidone. The experiment was carried out by adding 10 ml of the inoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media with increased viscosity by adding PVP contained nearly 3 times more DHA than those grown in a medium without increased PVP (FIG. 5).

### Example 6

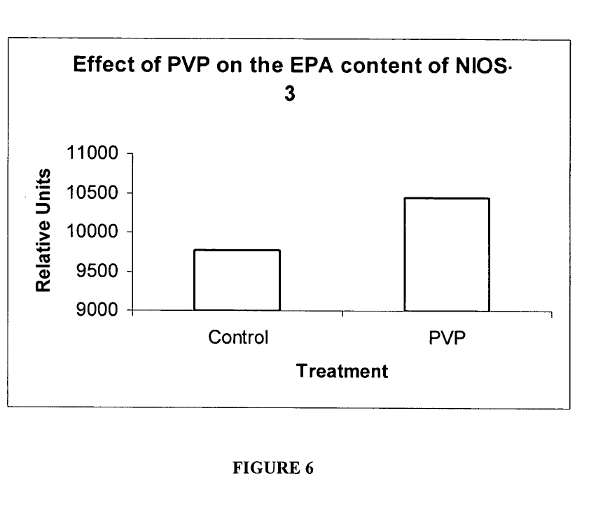

A culture of a thraustochytrid, belonging to strain #NIOS-3 was inoculated into 100 ml of a culture medium containing: gelatin peptone—1.5% Wt.; Yeast extract—0.1% Wt.; Glucose—1.0% wt. and sea water—100 ml. The cultures were grown for 2 days on a shaker at room temperature of 25-30 degree C. These cultures were used as inoculum for the experiment. A set of cultures was set up using a medium with the same composition as above, containing an addition of 1.0% polyvinyl pyrrolidone. The experiment was carried out by adding 10 ml of the inoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media with increased viscosity by adding PVP contained 2 times more EPA than that which was not stored in the refrigerator (FIG. 6).

### Example 7

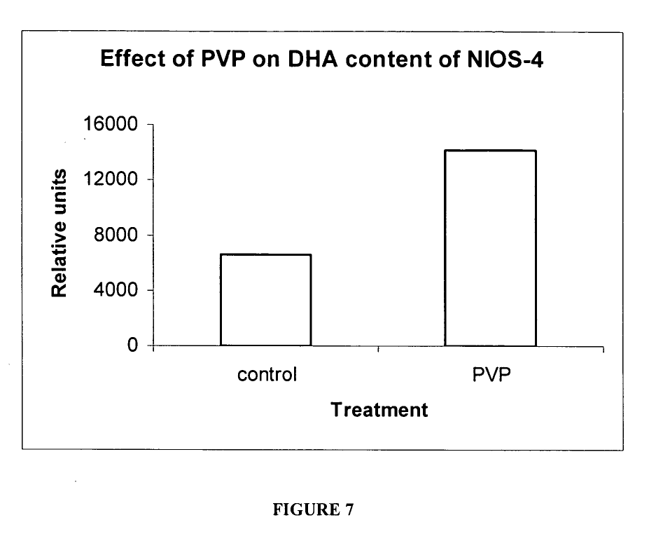

A culture of a thraustochytrid, belonging to strain #NIOS-4 was inoculated into 100 ml of a culture medium containing: gelatin peptone—1.5% Wt.; Yeast extract—0.1% Wt.; Glucose—1.0% Wt. and sea water—100 ml. The cultures were grown for 2 days on a shaker at room temperature of 25-30 degree C. These cultures were used as inoculum for the experiment. A set of cultures was set up using a medium with the same composition as above, containing an addition of 1.0% polyvinyl pyrrolidone. The experiment was carried out by adding 10 ml of the inoculum into 100 ml of the culture medium of the experimental set. The cultures were grown for 3 days on a shaker at room temperature of 25-30 degree C. At the end of this period, cells were harvested by centrifugation, fatty acids extracted and analyzed by gas chromatography. Cultures grown in media with increased viscosity by adding PVP contained nearly 3 times more DHA than those grown in a medium without increased PVP (FIG. 7).

The main advantages of the present invention are:
1. The DHA and EPA levels of thraustochytrids normally present in cultures can be further enhanced.
2. Even those strains that have only moderate quantities of DHA and EPA can be enriched in these fatty acids.
3. The viscosity of the medium is increased by addition of polyvinyl pyrrolidone, an easily available chemical.
4. Polyvinyl pyrrolidone is not used as nutrition by the cultures and, therefore, does not interfere with their normal metabolism.
5. Polyvinyl pyrrolidone is not toxic to the cultures and does not harm their normal metabolism.

1. A method for enhancing levels of docosahexaenoic acid and eicosapentaenoic acid in thraustochytrid protist, comprising the steps of:
(a) inoculating the thraustochytrid protist belonging to the genera Schizochytrium deposited at The Microbial Type Culture Collection (MTCC), Institute of Microbial Technology, Chandigarh, India under the accession number MTCC 5121 or Thraustochytrium deposited at The Microbial Type Culture Collection (MTCC), Institute of Microbial Technology, Chandigarh, India under the accession number MTCC 5122 or Aplanochytrium deposited at The Microbial Type Culture Collection (MTCC), Institute of Microbial Technology, Chandigarh, India under the accession number MTCC 5123 in a culture medium and growing the same for about 2 days at 25° C. to 30° C.;
(b) obtaining the cultures thus grown for use as inoculum and inoculating a medium having increased viscosity using the same;
(c) growing the thraustochytrid protist culture of step (b) for 2 to 5 days at 25° to 30° C., and
(d) harvesting the cells by centrifugation and extracting the enhanced amounts of docosahexaenoic acid and eicosapentaenoic acids from the cells.

2. A method as claimed in claim 1 wherein step (a), the culture medium used comprises peptone in the range of 0.5% Wt. to 1.5% Wt.; yeast extract in the range of 0.01% Wt. to 0.1% Wt.; glucose in the range of 0.01% to 1.0% Wt.; and sea water of about 100 ml.

3. A method as claimed in claim 1 wherein step (b), the culture medium used comprises peptone in the range of 0.5% Wt. to 1.5% Wt.; yeast extract in the range of 0.01% Wt. to 0.1% Wt.; glucose in the range of 0.01% to 1.0% Wt.; polyvinyl pyrrolidone in the range of 0.5% Wt. to 1.5% Wt.; and sea water of about 100 ml.

4. A method as claimed in claim 3, wherein the culture comprises 1.5% peptone; 0.1% yeast extract; 1.0% glucose; 1.0% polyvinyl pyrrolidone and 100 ml sea water.

5. A method according to claims 2 or 3, wherein said culture medium comprises 1.5% peptone.

6. A method according to claim 2 or 3, wherein said culture medium comprises 0.1% yeast extract.

7. A method according to claim 2 or 3, wherein said culture medium comprises 1.0% glucose.
